(19) United States
(12) Patent Application Publication
(10) Pub. No.: US 2017/0245916 A1
(43) Pub. Date: Aug. 31, 2017

(54) POWER SUPPLY DEVICE FOR HIGH FREQUENCY TREATMENT INSTRUMENT, AND TREATMENT SYSTEM INCLUDING THE SAME

(71) Applicant: OLYMPUS CORPORATION, Tokyo (JP)

(72) Inventor: Tateyuki SUGAWARA, Yokohama-shi (JP)

(73) Assignee: OLYMPUS CORPORATION, Tokyo (JP)

(21) Appl. No.: 15/595,281

(22) Filed: May 15, 2017

Related U.S. Application Data
(63) Continuation of application No. PCT/JP2016/063773, filed on May 9, 2016.

(30) Foreign Application Priority Data
Jun. 19, 2015 (JP) 2015-123623

(51) Int. Cl. A61B 18/12; A61B 18/16

(52) U.S. Cl. CPC A61B 18/1233; A61B 18/16; A61B 2018/00642

(57) ABSTRACT

A power supply device for a high frequency treatment instrument includes an active side detection circuit that acquires a first signal output from a treatment instrument-connecting terminal to the treatment instrument and a second signal returned from the treatment instrument to the treatment instrument-connecting terminal, a passive side detection circuit that acquires a third signal output from the treatment instrument-connecting terminal to the treatment instrument and passing through a return electrode to a return electrode-connecting terminal, and a processor that calculates a return loss as the second signal to the first signal and a first insertion loss as the third signal to the first signal and determines an abnormality occurrence location based thereon.

### CROSS-REFERENCE TO RELATED APPLICATIONS

This application is a Continuation application of PCT Application No. PCT/JP2016/063773, filed May 9, 2016 and based upon and claiming the benefit of priority from prior Japanese Patent Application No. 2015-123623, filed Jun. 19, 2015, the entire contents of all of which are incorporated herein by reference.

### BACKGROUND OF THE INVENTION

1. Field of the Invention

The present invention-relates to a power supply device for a high frequency treatment instrument, and a treatment system including the same.

2. Description of the Related Art

Treatment systems for treating living tissue with the use of high frequency power have been generally known. In such treatment systems, an electric knife is connected to one terminal of a high frequency power supply and a return electrode is connected to the other terminal. Treatment of living tissue with the treatment systems is performed while a high frequency current output from the electric knife is collected by the return electrode.

Such treatment systems monitor the occurrence of system abnormalities and, if there is an abnormality, issue a warning or halt the output of a high frequency power supply. For example, Jpn. Pat. Appln. KOKAI Publication No. 11-9611 discloses a technique to detect whether or not a return electrode is properly attached to a human body. According to this technique, whether or not a value of a detection signal, for example, impedance, is within a predetermined range is determined, and the value of the signal is stored if it is determined to be within the range. If a value of the detection signal varies from the stored value by a predetermined value, it is determined that the return electrode has been stripped off and a warning to indicate an abnormality is issued.

### BRIEF SUMMARY OF THE INVENTION

According to one embodiment of the present invention, a power supply device is a device for a treatment system to supply a high frequency electric current between an end electrode of a treatment instrument that uses high frequency power and a return electrode that is configured to be affixed to a surface of a human body. The power supply device includes a power source which generates alternating-current power; a treatment instrument-connecting terminal for electrically connecting the treatment instrument; a return electrode-connecting terminal for electrically connecting the return electrode; an active side detection circuit which acquires a first signal for the alternating-current power output from the power source to the treatment instrument via the treatment instrument-connecting terminal, and a second signal for a power output from the power source to the treatment instrument via the treatment instrument-connecting terminal and returned from the treatment instrument to the treatment instrument-connecting terminal; a passive side detection circuit which acquires a third signal for a power output from the power source to the treatment instrument via the treatment instrument-connecting terminal and passing through the return electrode to the return electrode-connecting terminal; and a processor which calculates a return loss as the second signal to the first signal and a first insertion loss as the third signal to the first signal, and determines an occurrence location of an abnormality based on the return loss and the first insertion loss upon occurrence of the abnormality in the treatment system.

According to one embodiment of the present invention, a treatment system includes the power supply device, the treatment instrument, and the return electrode.

Advantages of the invention will be set forth in the description which follows, and in part will be obvious from the description, or may be learned by practice of the invention. The advantages of the invention may be realized and obtained by means of the instrumentalities and combinations particularly pointed out hereinafter.

### BRIEF DESCRIPTION OF THE SEVERAL VIEWS OF THE DRAWINGS

The accompanying drawings, which are incorporated in and constitute a part of the specification, illustrate embodiments of the invention, and together with the general description given above and the detailed description of the embodiments given below, serve to explain the principles of the invention.

FIG. 1 shows one example of the external view of a treatment system according to a certain embodiment.

FIG. 2 is a block diagram schematically showing an exemplary configuration of a treatment system according to the first embodiment.

FIG. 3 shows one example of the circuit configuration of an active side detection circuit.

FIG. 4 is an illustration for explaining flows of signals in a treatment system according to the first embodiment.

FIG. 5 shows a table for explaining one example of the various parameters in a treatment system according to the first embodiment.

FIG. 6 shows a table for explaining one example of the method for identifying an abnormality occurrence location in a treatment system according to the first embodiment.

FIG. 7A is a flowchart showing one example of the processing of a power supply device.

FIG. 7B is a flowchart showing one example of the processing of a power supply device.

FIG. 8 is an illustration for explaining the operation of a power supply device and shows one example of the first return loss with respect to the passage of time.

FIG. 9 is an illustration for explaining the operation of the power supply device and shows one example of the first return loss with respect to the passage of time.

FIG. 10 is an illustration for explaining the operation of the power supply device and shows one example of the first return loss with respect to the passage of time.

FIG. 11 is an illustration for explaining the operation of the power supply device and shows one example of the first return loss with respect to the passage of time.

FIG. 12 is an illustration for explaining the operation of the power supply device and shows one example of the first return loss with respect to the passage of time.

FIG. 13 is an illustration for explaining the operation of the power supply device and shows one example of the first return loss with respect to the passage of time.

FIG. 14 is an illustration for explaining the operation of the power supply device and shows one example of the first return loss with respect to the passage of time.

FIG. 15 is an illustration for explaining the operation of the power supply device and shows one example of the first return loss with respect to the passage of time.

FIG. 16 is an illustration for explaining the operation of the power supply device and shows one example of the first return loss with respect to the passage of time.

FIG. 17 is an illustration for explaining the operation of the power supply device and shows one example of the first insertion loss with respect to the passage of time.

FIG. 18 is a block diagram schematically showing an exemplary configuration of a treatment system according to the second embodiment.

FIG. 19 shows a table for explaining one example of the various parameters in a treatment system according to the second embodiment.

FIG. 20 shows a table for explaining one example of the method for identifying an abnormality occurrence location in a treatment system according to the second embodiment.

### DETAILED DESCRIPTION OF THE INVENTION

### First Embodiment

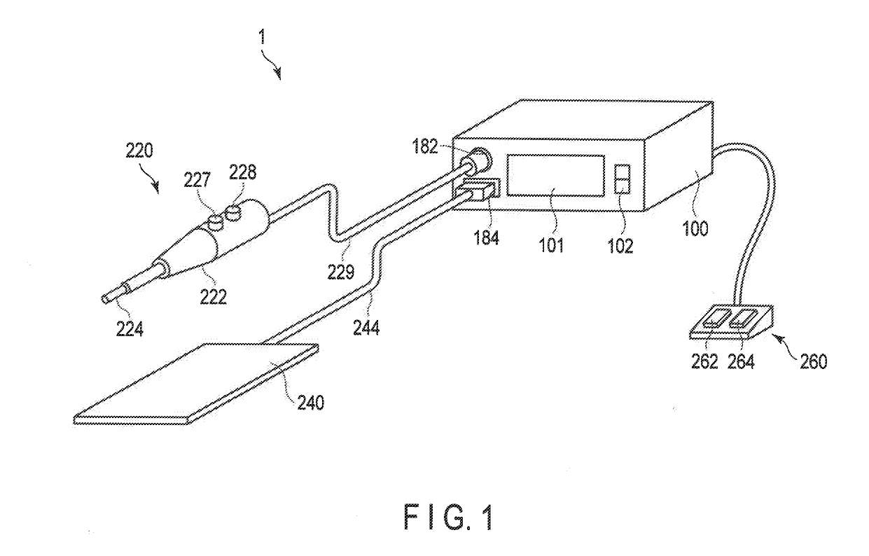

The first embodiment of the present invention will be described with reference to the drawings. FIG. 1 shows one example of the external view of a treatment system 1 according to this embodiment. As shown in FIG. 1, the treatment system 1 comprises a power supply device 100, a treatment instrument 220, a return electrode 240, and a foot switch 260.

One end of a first cable 229 is connected to the treatment instrument 220. The first cable 229 is a cable for the connection between the treatment instrument 220 and the power supply device 100. The other end of the first cable 229 is connected to a treatment instrument-connecting terminal 182 of the power supply device 100.

The treatment instrument 220 comprises an operation unit 222, an end electrode 224, a first switch 227, and a second switch 228. The operation unit 222 is a portion for a user to hold and to operate the treatment instrument 220. The end electrode 224 is provided at the distal end of the operation unit 222. During treatment, the end electrode 224 is applied to living tissue as a treatment subject.

The first switch 227 and the second switch 228 of the treatment instrument 220 are provided at the operation unit 222. The first switch 227 is a switch for an input to cause the power supply device 100 to output in an incision mode. The incision mode is a mode to burn and cut living tissue as a treatment subject at the portion contacting the end electrode 224, with a supply of relatively large electric power. The second switch 228 is a switch for an input to cause the power supply device 100 to output in a hemostasis mode. The hemostasis mode is a mode to perform hemostatic treatment, with a supply of electric power lower than in the incision mode, by burning and cutting living tissue as a treatment subject while denaturalizing an end section thereof at the portion contacting the end electrode 224.

The foot switch 260 comprises a first switch 262 and a second switch 264. The first switch 262 of the foot switch 260 has the same function as the first switch 227 provided at the treatment instrument 220. Also, the second switch 264 of the foot switch 260 has the same function as the second switch 228 provided at the treatment instrument 220. In other words, a user can perform on/off switching of the output of the treatment instrument 220 using the first switch 227 and the second switch 228 provided at the treatment instrument 220, and also using the first switch 262 and the second switch 264 of the foot switch 260.

The return electrode 240 is formed so that it may be affixed to a body surface of a patient to be treated. A second cable 244 is connected to the return electrode 240. The second cable 244 is a cable for the connection between the return electrode 240 and the power supply device 100. The second cable 244 is connected to a return electrode-connecting terminal 184 of the power supply device 100.

The power supply device 100 feeds electric power between the treatment instrument 220 and the return electrode 240. The power supply device 100 is provided with a display panel 101 and switches 102. The display panel 101 displays various information concerning the state of the power supply device 100. A user uses the switches 102 to input, for example, a setup value for the output such as output electric power, a setup value to define cutting performance called an effect, and so on, to the power supply device 100.

When the treatment system 1 is in use, a user as an operator brings the end electrode 224 into contact with a treatment subject site while, for example, pressing down the first switch 227 or the second switch 228 of the treatment instrument 220. At this time, an electric current output from the power supply device 100 flows between the end electrode 224 and the return electrode 240. As a result, incision or hemostasis of the living tissue is performed at the portion contacting the end electrode 224.

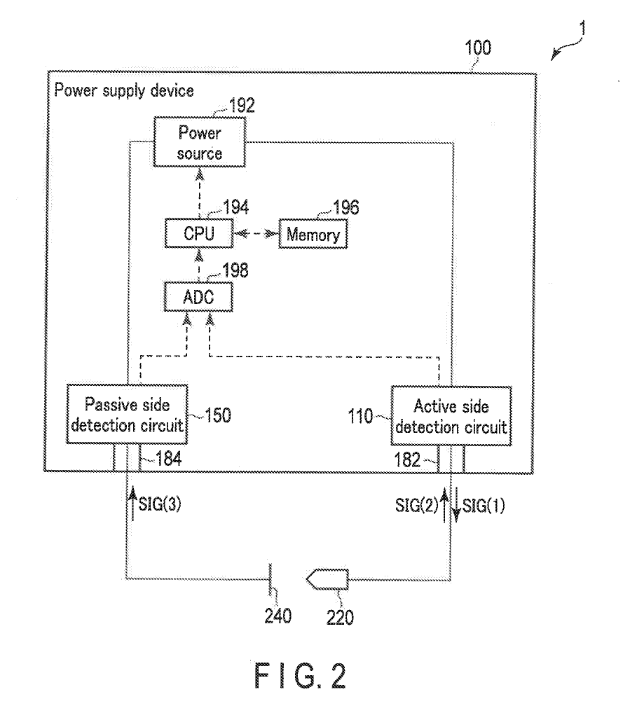

FIG. 2 schematically shows the configuration of the treatment system 1. The power supply device 100 comprises a power source 192, a central processing unit (CPU) 194, a memory 196, and an analog/digital converter (ADC) 198. The CPU 194 controls operations of each component of the power supply device 100 and performs various calculations. As such, the CPU 194 functions as a processor. The memory 196 stores programs and various parameters required for the operations of the CPU 194. The ADC 198 converts analog signals output from an active side detection circuit 110 and a passive side detection circuit 150, which will be described later, into digital signals and sends them to the CPU 194. The power source 192 takes power from the outside of the power supply device 100 and outputs alternating-current power in accordance with the calculation result of the CPU 194.

Near the treatment instrument-connecting terminal 182 connected to the treatment instrument 220, the active side detection circuit 110 is provided. The active side detection circuit 110 detects a first signal SIG(1) for the output power output from the treatment instrument-connecting terminal 182 of the power supply device 100 to the treatment instrument 220, and a second signal SIG(2) for the return power output from the treatment instrument-connecting terminal 182 to the treatment instrument 220 and returned from the treatment instrument 220 to the treatment instrument-connecting terminal 182. The first signal SIG(1) and the second signal SIG(2) are sent to the CPU 194 via the ADC 198. The first signal SIG(1) and the second signal SIG(2) may be amplified as appropriate.

Also, near the return electrode-connecting terminal 184 connected to the return electrode 240, the passive side detection circuit 150 is provided. The passive side detection circuit 150 detects a third signal SIG(3) for the power output from the treatment instrument-connecting terminal 182 of the power supply device 100 to the treatment instrument 220 and passing through the return electrode 240 to the return electrode-connecting terminal 184 of the power supply device 100. The third signal SIG(3) is sent to the CPU 194 via the ADC 198. The third signal SIG(3) may be amplified as appropriate.

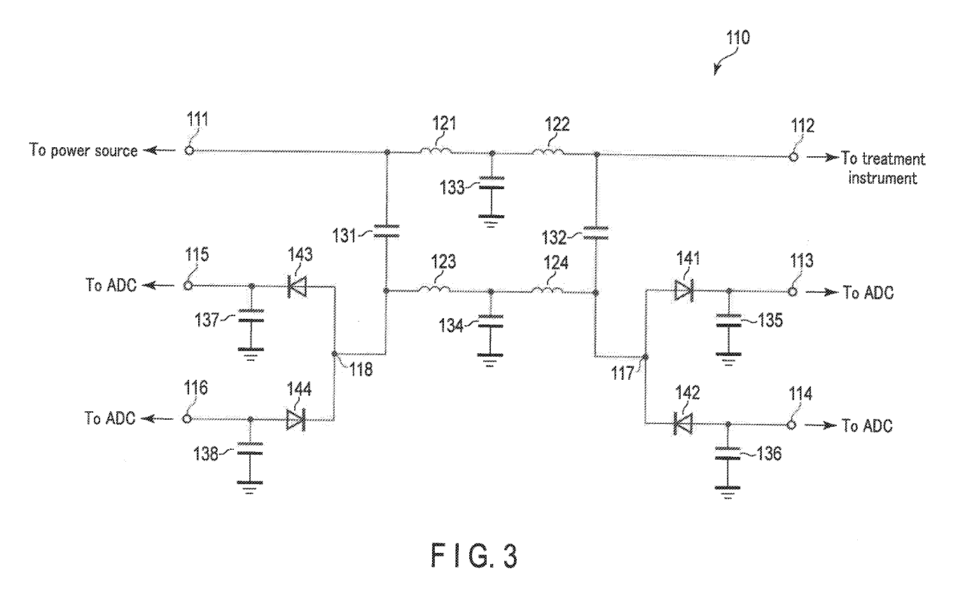

FIG. 3 shows one example of the circuit configuration of the active side detection circuit 110. As shown in FIG. 3, the active side detection circuit 110 is constituted by coils, capacitors, and diodes.

Of the terminals comprised by the active side detection circuit 110, the terminal into which an electric current output from the power source 192 will be referred to as a first terminal ill. Also, of the terminals comprised by the active side detection circuit 110, the terminal connected to the treatment instrument-connecting terminal 182 will be referred to as a second terminal 112. Also, of the two terminals for extracting the first signal SIG(1), one terminal will be referred to as a third terminal 113 and the other terminal will be referred to as a fourth terminal 114. Also, of the two terminals for extracting the second signal SIG(2), one terminal will be referred to as a fifth terminal 115 and the other terminal will be referred to as a sixth terminal 116.

Between the first terminal 111 and the second terminal 112, a first coil 121 and a second coil 122 are connected in series. The first coil 121 at its end on the first terminal 111 side is connected to one end of a first capacitor 131. The other end of the first capacitor 131 will be referred to as a second signal end 118. The second coil 122 at its end on the second terminal 112 side is connected to one end of a second capacitor 132. The other end of the second capacitor 132 will be referred to as a first signal end 117. Between the first coil 121 and the second coil 122, one end of a third capacitor 133 is connected. The other end of the third capacitor 133 is grounded.

Between the first signal end 117 and the second signal end 118, a third coil 123 and a fourth coil 124 are connected in series. Between the third coil 123 and the fourth coil 124, one end of a fourth capacitor 134 is connected. The other end of the fourth capacitor 134 is grounded.

An anode of a first diode 141 is connected to the first signal end 117. A cathode of the first diode 141 is connected to the third terminal 113. The cathode of the first diode 141 is also connected to one end of a fifth capacitor 135. The other end of the fifth capacitor 135 is grounded. With the fifth capacitor 135, charge-voltage conversion is performed so that a signal voltage may be extracted from the third terminal 113.

Also, a cathode of a second diode 142 is connected to the first signal end 117. An anode of the second diode 142 is connected to the fourth terminal 114. The anode of the second diode 142 is also connected to one end of a sixth capacitor 136. The other end of the sixth capacitor 136 is grounded. With the sixth capacitor 136, charge-voltage conversion is performed so that a signal voltage may be extracted from the fourth terminal 114.

An anode of a third diode 143 is connected to the second signal end 118. A cathode of the third diode 143 is connected to the fifth terminal 115. The cathode of the third diode 143 is also connected to one end of a seventh capacitor 137. The other end of the seventh capacitor 137 is grounded. With the seventh capacitor 137, charge-voltage conversion is performed so that a signal voltage may be extracted from the fifth terminal 115.

Also, to the second signal end 118, a cathode of a fourth diode 144 is connected. An anode of the fourth diode 144 is connected to the sixth terminal 116. The anode of the fourth diode 144 is also connected to one end of an eighth capacitor 138. The other end of the eighth capacitor 138 is grounded. With the eighth capacitor 138, charge-voltage conversion is performed so that a signal voltage may be extracted from the sixth terminal 116.

As described above, the first terminal 111 and the second terminal 112 are configured to be symmetric with each other. Also, the first signal end 117 and the second signal end 118 are configured to be symmetric with each other. As a matter of course, the circuit configuration shown in FIG. 3 represents one embodiment and does not limit the present invention, and other configurations including asymmetric arrangements may be adopted based on this circuit configuration. According to the circuit configuration, the third terminal 113 allows for the acquisition of an intensity of positive signals among the signals having a correlation with signals passing from the first terminal 111 to the second terminal 112. Also, the fourth terminal 114 allows for the acquisition of an intensity of negative signals among the signals having a correlation with signals passing from the first terminal 111 to the second terminal 112. Likewise, the fifth terminal 115 allows for the acquisition of an intensity of positive signals among the signals having a correlation with signals passing from the second terminal 112 to the first terminal 111. Also, the sixth terminal 116 allows for the acquisition of an intensity of negative signals among the signals having a correlation with signals passing from the second terminal 112 to the first terminal 111.

When in FIG. 2, each terminal shown in FIG. 3 has the following connection relationship. The first terminal 111 is connected to the power source 192. The second terminal 112 is connected to the treatment instrument 220 via the treatment instrument-connecting terminal 182. The third terminal 113 and the fourth terminal 114 are connected to the ADC 198. Also, the fifth terminal 115 and the sixth terminal 116 are connected to the ADC 198.

As such, the active side detection circuit 110 is for the acquisition of the signals having a correlation with the signals (main signals) passing along the path between the first terminal 111 and the second terminal 112, from the third terminal 113 and the fourth terminal 114, and from the fifth terminal 115 and the sixth terminal 116. Note that the signals acquired from the third terminal 113 and the fourth terminal 114, and from the fifth terminal 115 and the sixth terminal 116 are generally smaller than the main signals. Also, the subject of the signal detection is electric power. This electric power is converted into an analog voltage signal between the first signal end 117 and the third terminal 113 or the fourth terminal 114. Likewise, the electric power is converted into an analog voltage signal between the second signal end 118 and the fifth terminal 115 or the sixth terminal 116. This analog voltage signal will be converted into a digital signal at the ADC 198.

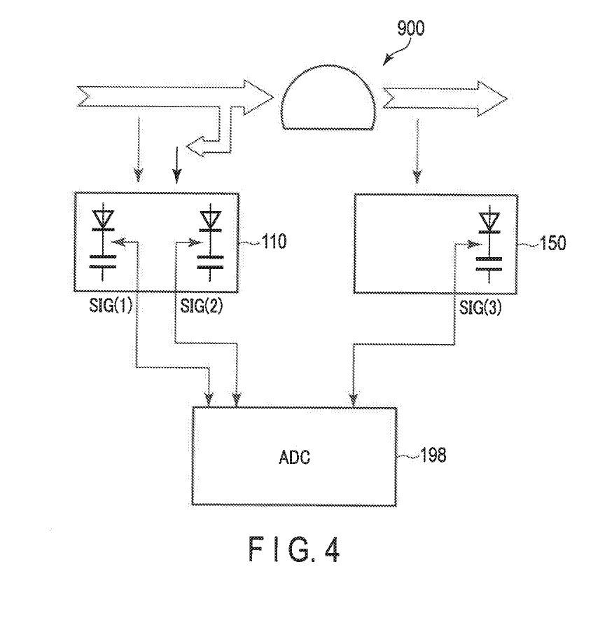

FIG. 4 schematically illustrates the electric power passing through a patient 900 as the treatment subject, and the obtained signals. As shown by the white arrows in FIG. 4, electric power is supplied to the patient 900 and mostly passes through the patient 900, with some being partly reflected. The active side detection circuit 110 acquires a signal corresponding to the electric power input to the patient 900 as the first signal SIG(1). The first signal SIG(1) is sent to the ADC 198. The active side detection circuit 110 also acquires a signal corresponding to the electric power returned from the patient 900 as the second signal SIG(2). The second signal SIG(2) is sent to the ADC 198. Also, the passive side detection circuit 150 acquires a signal corresponding to the electric power having passed through the patient 900 as the third signal SIG(3). The third signal SIG(3) is sent to the ADC 198.

The passive side detection circuit 150 has a circuit configuration similar to the active side detection circuit 110. In the circuit similar to the circuit shown in FIG. 3, the power source 192 is connected to a terminal corresponding to the first terminal 111 and the return electrode-connecting terminal 184 is connected to a terminal corresponding to the second terminal 112. Also, terminals corresponding to the fifth terminal 115 and the sixth terminal 116 are adapted to extract the third signal SIG(3) and are connected to the ADC 198.

As described above, the active side detection circuit 110 and the passive side detection circuit 150 are provided with the terminals for signal detection. Each of these terminals would involve mixed signals, which may be addressed by setting the thresholds described later so that the thresholds take into account the mixed signals.

Next, the information obtained based on the first signal SIG(1), the second signal SIG(2), and the third signal SIG(3) will be described.

The second signal SIG(2) to the first signal SIG(1) is defined as a first return loss RL(1). That is, the first return loss is given as:

RL(1)=SIG(2)/SIG(1).

The first return loss RL(1) indicates how much electric power is returned from the treatment instrument 220 side for the output from the power supply device 100; that is, a reflection rate. Therefore, if the first return loss RL(1) is large, it can be assumed that an electric current is not properly flowing into the treatment instrument 220 due to, for example, a disconnection between the treatment instrument-connecting terminal 182 of the power supply device 100 and the treatment instrument 220. Also, when the end electrode 224 of the treatment instrument 220 is not in contact with living tissue as a treatment subject, the first return loss RL(1) increases.

The third signal SIG(3) to the first signal SIG(1) is defined as a first insertion loss IL(1). That is, the first insertion loss is given as:

IL(1)=SIG(3)/SIG(1).

The first insertion loss IL(1) indicates how much electric power output from the power supply device 100 passes through the treatment instrument 220, the patient 900, and the return electrode 240 to enter the power supply device 100, that is, a passage rate. Therefore, if the first insertion loss IL(1) is small, it can be assumed, for example, that there is a large leakage current to an operator or other devices.

As described above, based on the first signal SIG(1) and the second signal SIG(2), an abnormality in the path between the treatment instrument-connecting terminal 182 and the treatment instrument 220 can be detected immediately upon occurrence. Also, based on the first signal SIG(1) and the third signal SIG(3), an abnormality in the path from the treatment instrument-connecting terminal 182 to the return electrode-connecting terminal 184 via the treatment instrument 220, the patient 900, and the return electrode 240 can be detected immediately upon occurrence. Moreover, since changes from the normal state can be ascertained even in the course of an abnormality occurring, the output can be controlled also before an abnormality.

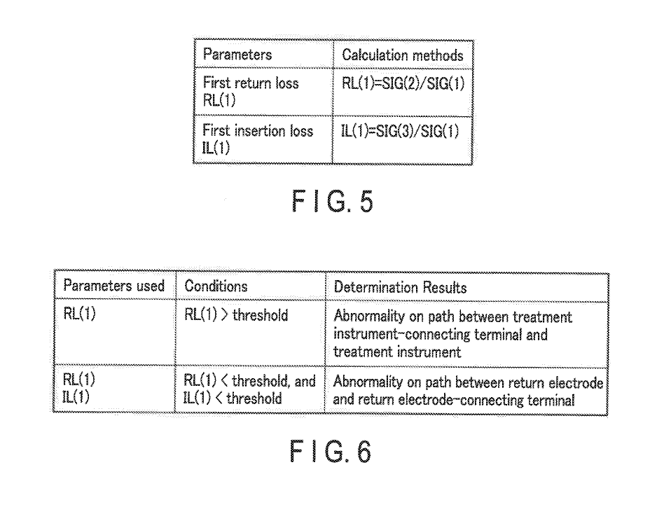

The above descriptions may be summarized as shown in FIG. 5 and FIG. 6. That is, as shown in FIG. 5, the first return loss RL(1) is obtained as RL(1)=SIG(2)/SIG(1) and the first insertion loss IL(1) is obtained as IL(1)=SIG(3)/SIG(1). Also, as shown in FIG. 6 by way of example, if the first return loss RL(1) is larger than a predetermined threshold, an abnormality can be located on the path between the treatment instrument-connecting terminal 182 and the treatment instrument 220. Also, if the first return loss RL(1) is smaller than a predetermined threshold and the first insertion loss IL(1) is smaller than a predetermined threshold, an abnormality can be located on the path between the return electrode 240 and the return electrode-connecting terminal 184.

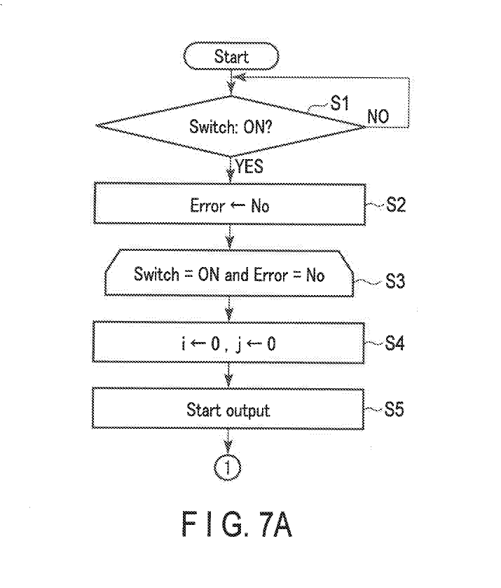

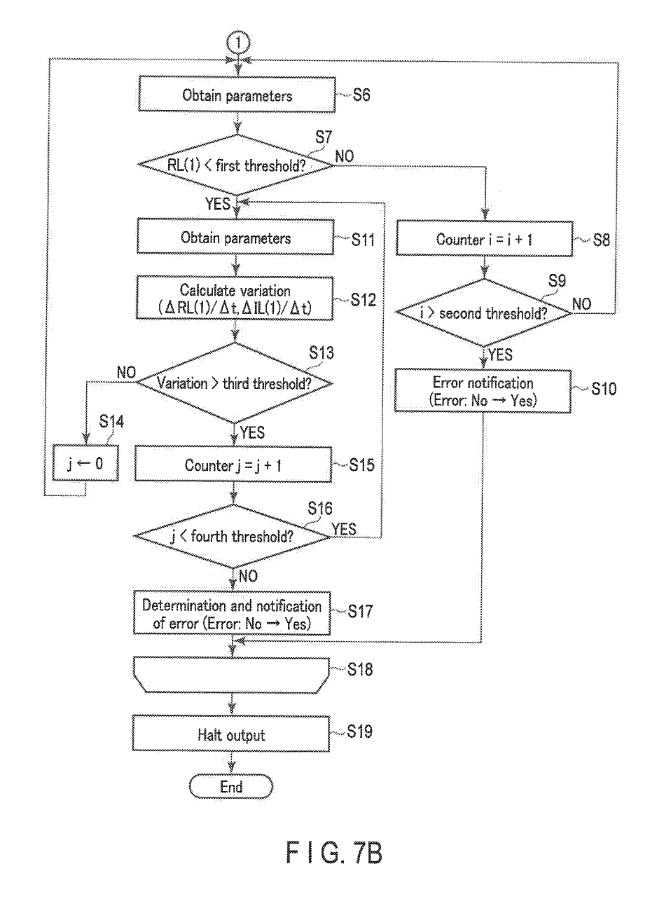

Next, the operations of the power supply device 100 according to this embodiment will be described with reference to the flowcharts shown in FIG. 7A and FIG. 7B. The processing starts upon, for example, turning on of the power supply device 100.

In step S1, the CPU 194 makes a determination about the signal of an output switch. The output switch here may be the first switch 227 and the second switch 228 of the treatment instrument 220 or the first switch 262 and the second switch 264 of the foot switch 260. The switch signal being ON represents a state where an operational input for feeding electric power between the treatment instrument 220 and the return electrode 240 has been done. If the switch signal is OFF, the processing returns to step S1. That is, the processing is placed in a loop. On the other hand, if the switch signal is ON, the processing proceeds to step S2.

In step S2, the CPU 194 initializes variables. For example, the CPU 194 initializes a variable, i.e., an error signal, so that it indicates No. The error signal is a variable to indicate whether or not there is an error.

The processing from step S3 to step S18 is repetitive processing. The conditions for repeating the processing from step S3 to step S18 are that: (1) the switch signal is ON, and (2) the error signal indicates No. When the switch signal is OFF, or when the error signal indicates Yes, the processing comes out of this repetitive processing and proceeds to step S19.

In step S4, the CPU 194 initializes a counter i to zero. The counter i is used for the determination in step S9. Also, the CPU 194 initializes a counter j to zero. The counter j is used for the determination in step S16.

In step S5, the CPU 194 causes the power source 192 to start outputting. The intensity of the output electric power here is based on a value set by a user. It may vary depending on which of the first switch 227 and the second switch 228 of the treatment instrument 220 has been pressed.

In step S6, the CPU 194 obtains various parameters. The parameters obtained here include at least the first signal SIG(1), the second signal SIG(2), and the third signal SIG(3). Also, the CPU 194 calculates the first return loss RL(1) and the first insertion loss IL(1) based on these signals.

In step S7, the CPU 194 determines whether or not the first return loss RL(1) is smaller than a predetermined first threshold. The first threshold here, which will be described in more detail later, is set to a value larger than the value obtained when the end electrode 224 of the treatment instrument 220 gets closer to the living tissue of the patient 900 as a treatment subject. That is, the first return loss RL(1) being smaller than the predetermined first threshold means that the end electrode 224 of the treatment instrument 220 has-been brought closer to the living tissue of the patient 900 as a treatment subject and the incision or hemostasis of the treatment subject is being carried out. If the first return loss RL(1) is not smaller than the first threshold, the processing proceeds to step S8. Note that, for this determination, determining whether or not the first insertion loss IL(1) is larger than a predetermined threshold may also be adopted instead of determining whether or not the first return loss RL(1) is smaller than the predetermined first threshold.

In step S8, the CPU 194 incrementally increases the count of the counter i.

In step S9, the CPU 194 determines whether or not the counter i is larger than a predetermined second threshold. If the counter i is not larger than the second threshold, the processing returns to step S6. On the other hand, if the counter i is larger than the second threshold, the processing proceeds to step S10.

Here, this proceeding to step S10 occurs in a state where the first return loss RL(1) remains large for a long time despite the switch being ON. This state corresponds to, for example, a scenario in which the end electrode 224 of the treatment instrument 220 is not brought closer to the treatment subject even though the switch has been turned ON. Also, such a state is possible when, for example, there is a disconnection between the power supply device 100 and the end electrode 224 of the treatment instrument 220. In this embodiment, a notification of a timeout error is issued in such cases. The second threshold may be set to a value that matches with the timing to issue a notification of a timeout error.

In step S10, the CPU 194 performs error notification. The manner of error notification includes output of an error sound, display of an error indication on a monitor, and so on. Also, the status of the error signal is changed from No to Yes. Then the processing proceeds to step S18. Since the error signal indicates Yes, the repetitive processing from step S3 to step S18 is terminated and the processing proceeds to step S19.

In step S7, if the first return loss RL(1) is determined to be smaller than the first threshold, the processing proceeds to step S11. In step S11, the CPU 194 obtains various parameters again, as they will be required in the processing in the next step S12. The parameters obtained here are the first signal SIG(1), the second signal SIG(2), and the third signal SIG(3). Also, the CPU 194 calculates the first return loss RL(1) and the first insertion loss IL(1) based on these signals.

In step S12, the CPU 194 calculates the variation of the first return loss RL(1) per unit of time. This variation is a value obtained by dividing the difference between the first return loss RL(1) obtained in step S6 and the first return loss RL(1) obtained in step S11 by the amount of time therebetween, that is, ΔRL(1)/Δt. The CPU 194 may also calculate the variation of the first insertion loss IL(1) per unit of time. This variation is a value obtained by dividing the difference between the first insertion loss IL(1) obtained in step S6 and the first insertion loss IL(1) obtained in step S11 by the amount of time therebetween, that is, ΔIL(1)/Δt.

In step S13, the CPU 194 determines whether or not the variation is larger than a predetermined third threshold. If the variation is not larger than the third threshold, the processing proceeds to step S14. In step S14, the CPU 194 initializes the counter j to zero. The processing then returns to step S6.

The determination in step S13 is for the estimation of whether the increase in the first return loss RL(1) or the decrease in the first insertion loss IL(1) is due to an abnormality of the treatment system 1, or due to an operator's manipulation activity of separating the treatment instrument 220 from the treatment subject. For example, when an abnormality of the treatment system 1 occurs, such as disconnection within the treatment instrument 220, variation of the first return loss RL(1) or the first insertion loss IL(1) per unit of time becomes significant. On the other hand, the variation of the first return loss RL(1) or the first insertion loss IL(1) per unit of time due to the operator's manipulation activity is not as large as in the cases of abnormality occurrences. The third threshold is set to a value which allows the estimation of whether the change in the first return loss RL(1) or the first insertion loss IL(1) is due to an abnormality of the treatment system 1, or due to a manipulation activity.

In step S13, if the variation is determined to be larger than the third threshold, the processing proceeds to step S15. In step S15, the CPU 194 incrementally increases the count of the counter j.

In step S16, the CPU 194 determines whether or not the counter j is smaller than a predetermined fourth threshold. If the counter j is smaller than the fourth threshold, the processing returns to step S11. Accordingly, when the counter j is smaller than the fourth threshold, the processing from step S11 to step S16 is repeated. On the other hand, if the counter j is not smaller than the fourth threshold, the processing proceeds to step S17. The determination in step S16 is for preventing a determination that an error has been detected when the variation exceeds the third threshold due to noise. When an abnormality occurs, the state of, for example, a very large first return loss RL(1) continues. However, noise may also often increase the first return loss RL(1) to a very large extent, but this increase mostly recedes in a short time. The fourth threshold is set to a value which allows the estimation of whether or not the variation exceeding the third threshold is due to noise. With this, an unintended error determination due to noise can be prevented.

In step S17, the CPU 194 determines a causative location for the error occurrence and performs error notification. The causative location is determined in accordance with the conditions described with reference to FIG. 6. As the manner of error notification, output of an error sound, display of an error indication on a monitor, or the like may be adopted. Also, the status of the error signal is changed from No to Yes. Since the error signal indicates Yes, the repetitive processing from step S3 to step S18 is terminated and the processing proceeds to step S19.

In step S19, the CPU 194 stops the output of the power source 192. The processing ends.

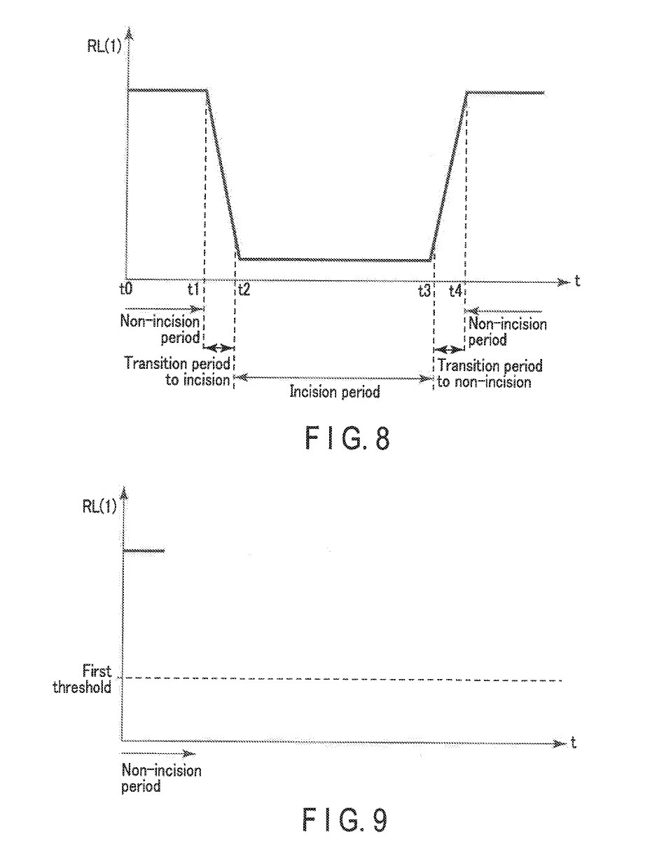

Referring to FIG. 8 to FIG. 16, the relationship between time t for an operator performing treatment and the first return loss RL(1) will be described.

FIG. 8 shows the relationship between time t for performing general treatment and the first return loss RL(1). As shown in FIG. 8, a treatment subject and the end electrode 224 of the treatment instrument 220 are sufficiently distant from each other during a period from time t0 to time t1, so the first return loss RL(1) shows a large value. This period represents a non-incision period where the treatment of a treatment subject, such as incision or hemostasis, is not performed.

As the end electrode 224 is brought closer to the treatment subject, an electric discharge, etc. occurs between the treatment subject and the end electrode 224 and an electric current flows. During this time, the first return loss RL(1) gradually decreases. A period from time t1 to time t2 represents a transition period from the non-incision period to an incision period.

When the treatment subject and the end electrode 224 get close to or contact each other, incision or hemostasis of the treatment subject is performed. During this time, the first return loss RL(1) is a small value. A period from time t2 to time t3 where the first return loss RL(1) is small represents the incision period.

As the end electrode 224 is moved away from the treatment subject, the first return loss RL(1) gradually increases. A period from time t3 to time t4 represents a transition period from the incision period to a non-incision period. A period from time t4 and onward represents a non-incision period. In the non-incision period, the value of the first return loss RL(1) is large.

Operations after turning ON the switch will be described in detail with respect to the passage of time. As shown in FIG. 9, the value of the first return loss RL(1) is larger than the first threshold during the non-incision period. Thus, during this time, the determination of NO is made in the processing of step S7. If this state continues for a certain duration, that is, for a period for the counter i to exceed the second threshold, the error notification in step S10 is performed. Such a timeout error may occur, for example, in the instances in which the return electrode 240 is not properly affixed to the body of the patient 900. Therefore, thanks to the timeout error notification, starting an operation without the return electrode 240 appropriately affixed to the body of the patient 900 can be prevented.

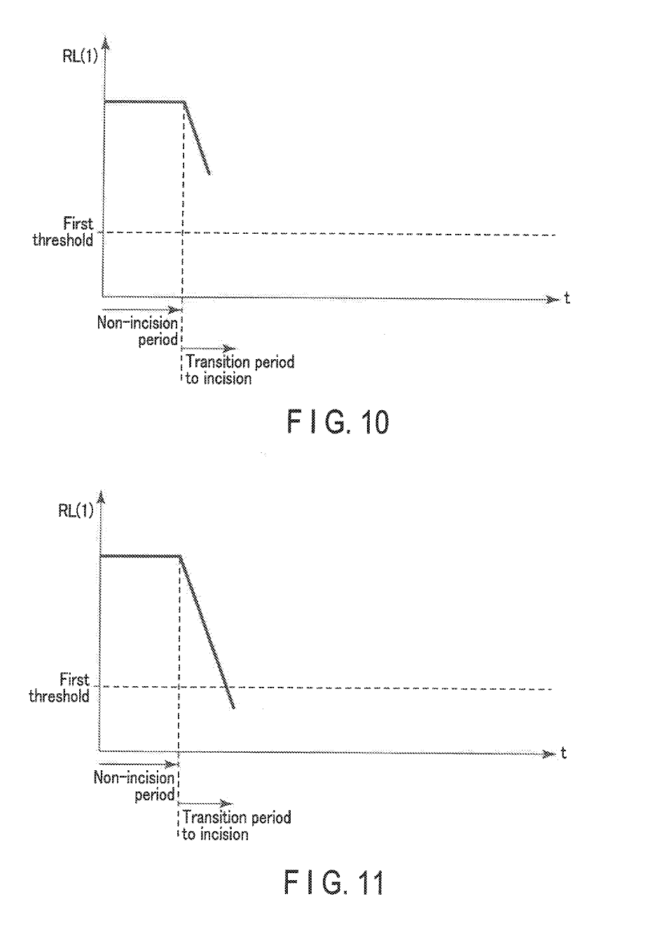

FIG. 10 shows the period for the transition to incision. During this time, the value of the first return loss RL(1) decreases due to the electric discharge, etc. between the treatment subject and the end electrode 224. For the state shown in FIG. 10, the first return loss RL(1) is larger than the first threshold. Thus, during this time, the determination of NO is made in step S7.

FIG. 11 shows the period for the transition to incision. During this time, the value of the first return loss RL(1) further decreases from the case shown in FIG. 10, due to the electric discharge, etc. between the treatment subject and the end electrode 224. For the state shown in FIG. 11, the first return loss RL(1) is smaller than the first threshold. Thus, during this time, the determination of YES is made in step S7. Accordingly, the processing proceeds to step S11.

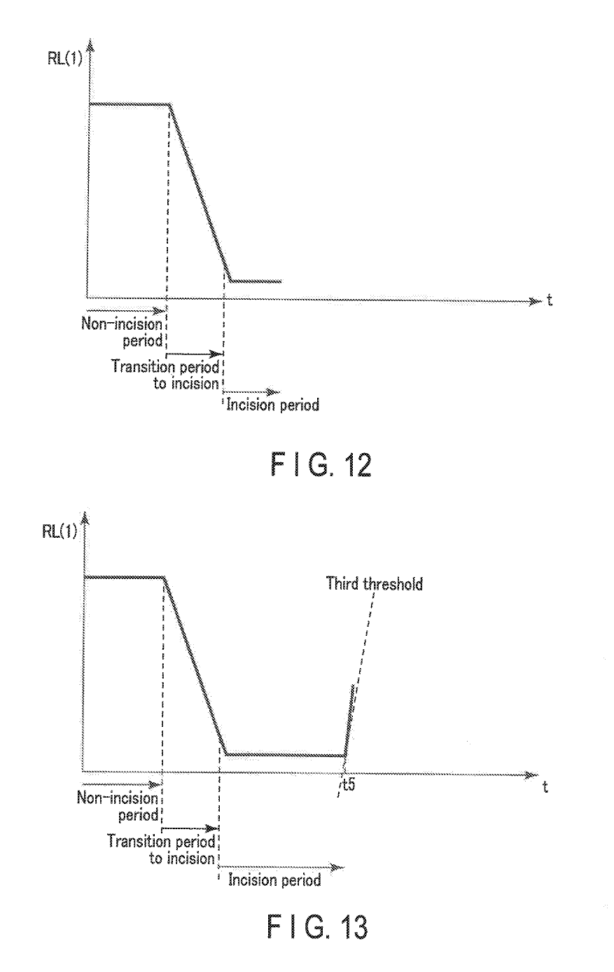

FIG. 12 shows the period for incision. As the figure has been simplified, the pattern of the first return loss RL(1) is drawn straight. However, the value of the first return loss RL(1) in practice fluctuates to some extent.

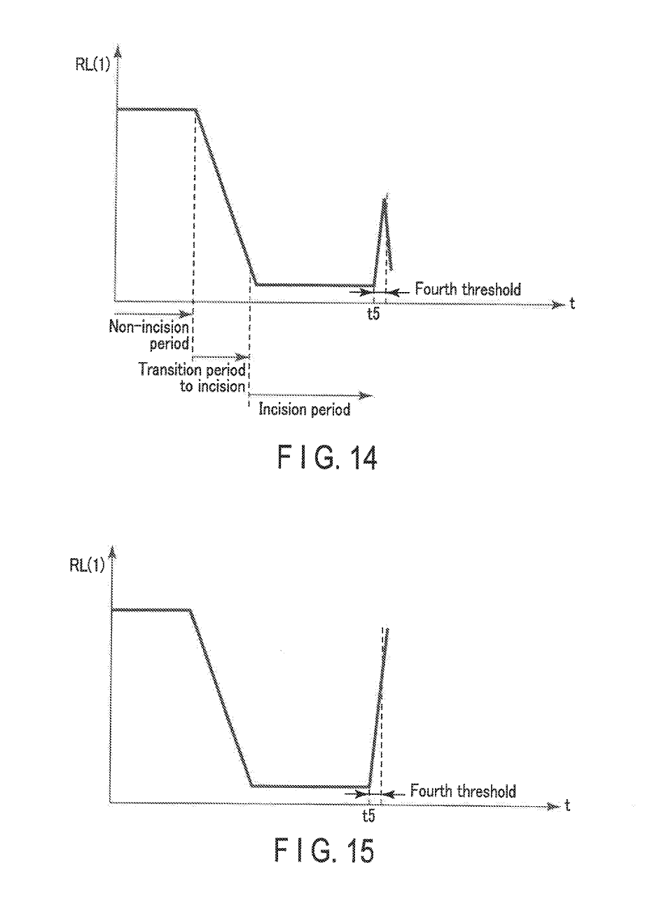

FIG. 13 shows an instance where an abnormality occurs in the treatment system 1 during the incision. Here, it is assumed that the abnormality is present from time t5. In the instance shown in FIG. 13, the variation of the first return loss RL(1) per unit of time is larger than the third threshold. At this time, the determination of YES is made in step S13. That is, the processing proceeds to step S15 to incrementally increase the counter j. In the instance shown in FIG. 14, the counter j, which is indicative of a time the variation has exceeded the third threshold, is smaller than the fourth threshold. Thus, at this time, the determination of YES is made in step S16. Note that when the variation becomes equal to or smaller than the third threshold, the counter j is initialized to zero in the processing in step S14.

In the instance shown in FIG. 15, the counter j, which is indicative of a time the variation has exceeded the third threshold, is larger than the fourth threshold. At this time, the determination of NO is made in step S16. The processing then proceeds to step S17 to perform error notification.

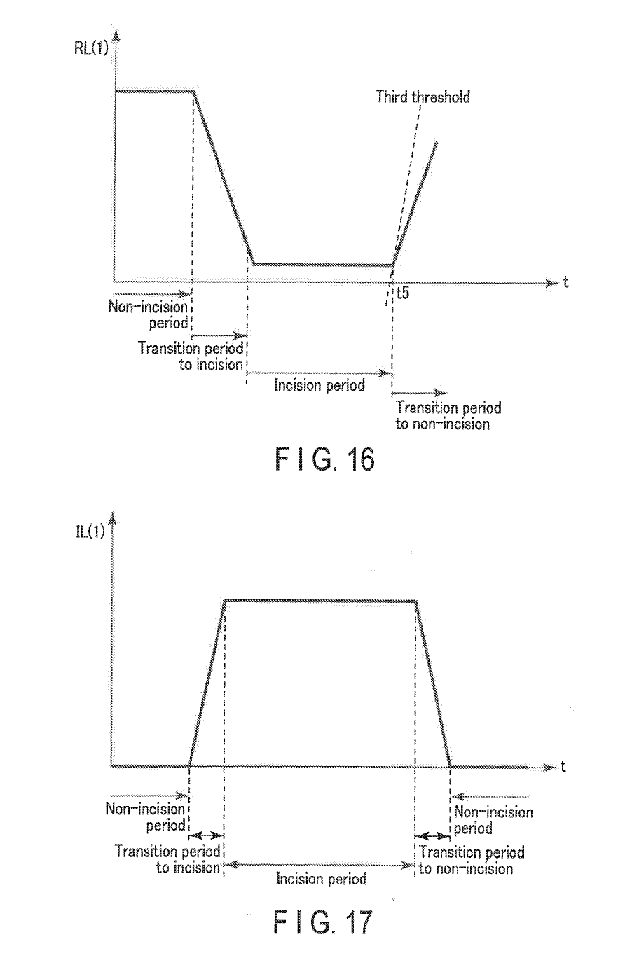

FIG. 16 shows an instance where the operator has moved the end electrode 224 away from the patient 900 in the manipulation activity of the treatment. During the period of transition from the incision period after time t5 to the non-incision period, the first return loss RL(1) increases. In terms of the increase in the first return loss RL(1), this is similar to the instance shown in FIG. 13 (when the instance of FIG. 15 follows). However, in FIG. 16, the variation of the first return loss RL(1) per unit of time is smaller than the third threshold, and therefore, an error notification is not performed. That is, estimating a reason for the increase in the first return loss RL(1), i.e., estimating whether the increase is due to the occurrence of an abnormality or due to a manipulation activity, has already been accomplished, and an unintended error notification during the manipulation activity state can be avoided.

Note that the descriptions above have been made with particular reference to the first return loss RL(1). However, the first insertion loss IL(1) is roughly in an inverse relationship with the first return loss RL(1) as shown in FIG. 17. Therefore, a similar estimation is possible by, for example, replacing the determination as to whether or not the first return loss RL(1) is smaller than a predetermined threshold with the determination as to whether or not the first insertion loss IL(1) is larger than a predetermined threshold. Similar processing may also be adopted in relation to the determination of a variation of the first insertion loss IL(1).

According to this embodiment, when an abnormality occurs in the treatment system 1, the occurrence location of this abnormality can be specified in the manner as above. That is, it is possible to specify if the abnormality has occurred due to, for example, a disconnection, etc. between the power supply device 100 and the treatment instrument 220, or a disconnection, etc. between the power supply device 100 and the return electrode 240.

The active side detection circuit 110 and the passive side detection circuit 150 may be constituted only by coils and capacitors. With this, the active side detection circuit 110 and the passive side detection circuit 150 can detect the signals required for abnormality detection while minimizing the loss of the energy for incision, hemostasis, etc.

### Second Embodiment

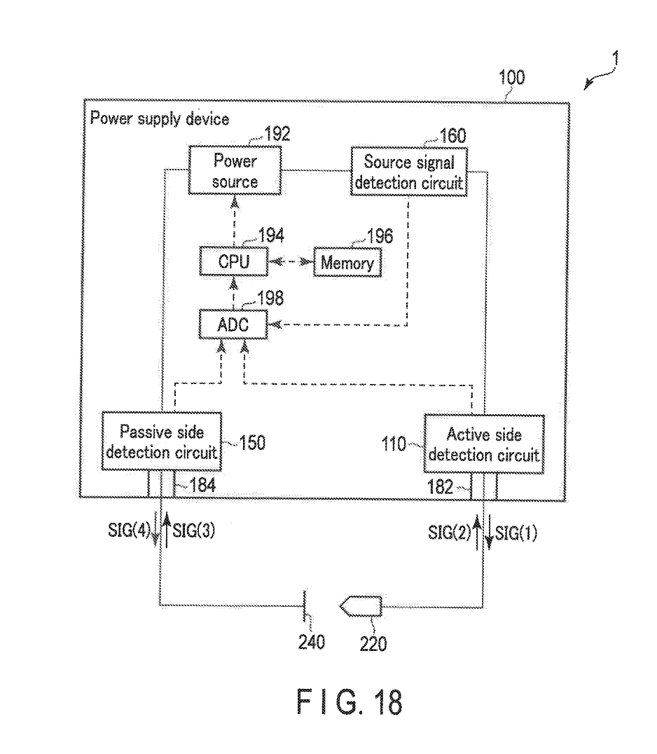

The second embodiment will be described. Hereinafter, differences from the first embodiment will be explained, and the elements similar to previously-described elements will be denoted by the same reference symbols and redundant descriptions will basically be omitted. FIG. 18 shows an overview of the configuration example of the power supply device 100 according to this embodiment. In this embodiment, a source signal detection circuit 160 is provided near the power source 192 on the path from the power source 192 to the active side detection circuit 110. The source signal detection circuit 160 is for acquiring a source signal SIG(0) output from the power source 192. Similar to the active side detection circuit or the passive side detection circuit, the source signal detection circuit 160 acquires, as a voltage signal, an electric power having a correlation with the electric power output from the power source 192. The source signal detection circuit 160 may also be provided between the power source 192 and the passive side detection circuit 150. The source signal SIG(0) is sent to the CPU 194 via the ADC 198.

Also, in addition to the third signal SIG(3), the passive side detection circuit 150 according to this embodiment detects a fourth signal SIG(4) for the power output from the return electrode-connecting terminal 184 of the power supply device 100 to the return electrode 240. The fourth signal SIG(4) is sent to the CPU 194 via the ADC 198. The other configurations of the treatment system 1 are similar to those for the first embodiment.

The power supply device 100 according to this embodiment obtains the first return loss RL(1) and the first insertion loss IL(1) as in the first embodiment. The power supply device 100 according to this embodiment further obtains a second insertion loss IL(2) and a third insertion loss IL(3) as set forth next and determines the state of the treatment system 1.

The second insertion loss IL(2) is the first signal SIG(1) to the source signal SIG(0). That is, the second insertion loss is given as:

IL(2)=SIG(1)/SIG(0).

The second insertion loss IL(2) indicates a rate of passage of the power within the power supply device 100. As such, when the second insertion loss IL(2) is small, it can be assumed that there is a malfunction between the power source 192 and the active side detection circuit 110 within the power supply device 100.

The third insertion loss IL(3) is the fourth signal SIG(4) to the source signal SIG(0). That is, the third insertion loss is given as:

IL(3)=SIG(4)/SIG(0).

The third insertion loss IL(3) indicates a rate of passage of the power within the power supply device 100. As such, when the third insertion loss IL(3) is small, it can be assumed that there is a malfunction between the power source 192 and the passive side detection circuit 150 within the power supply device 100.

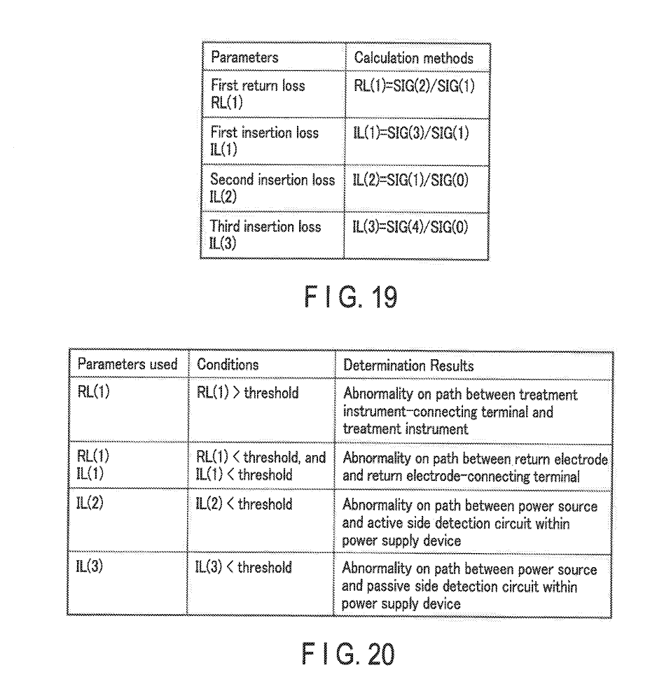

The above descriptions may be summarized as shown in FIG. 19 and FIG. 20. That is, as shown in FIG. 19, the first return loss RL(1) is obtained as RL(1)=SIG(2)/SIG(1), the first insertion loss IL(1) is obtained as IL(1)=SIG(3)/SIG(1), the second insertion loss IL(2) is obtained as IL(2)=SIG(1)/SIG(0), and the third insertion loss IL(3) is obtained as IL(3)=SIG(4)/SIG(0). Also, as shown in FIG. 20, if the first return loss RL(1) is larger than a predetermined threshold, an abnormality can be located on the path between the treatment instrument-connecting terminal 182 and the treatment instrument 220. Also, if the first return loss RL(1) is smaller than a predetermined threshold and the first insertion loss IL(1) is smaller than a predetermined threshold, an abnormality can be located on the path between the return electrode 240 and the return electrode-connecting terminal 184. Also, if the second insertion loss IL(2) is smaller than a predetermined threshold, an abnormality can be located on the path between the power source 192 and the active side detection circuit 110. Also, if the third insertion loss IL(3) is smaller than a predetermined threshold, an abnormality can be located on the path between the power source 192 and the passive side detection circuit 150.

Operations of the power supply device 100 according to this embodiment are basically the same as the operations of the power supply device 100 according to the first embodiment described with reference to FIG. 7A and FIG. 7B. Differences from the first embodiment will be explained.

The parameters obtained by the CPU 194 in step S6 and step S11 include the fourth signal SIG(4) and the source signal SIG(0), in addition to the first signal SIG(1), the second signal SIG(2), and the third signal SIG(3). Based on these signals, the CPU 194 calculates the second insertion loss IL(2) and the third insertion loss IL(3), in addition to the first return loss RL(1) and the first insertion loss IL(1).

Variations calculated by the CPU 194 in step S12 include IL(2)/Δt and IL(3)/Δt, in addition to ΔRL(1)/Δt and ΔIL(1)/Δt. In step S17, based on these calculation results, the CPU 194 determines a causative location for the error occurrence in accordance with the conditions described with reference to FIG. 20 and performs error notification.

According to this embodiment, abnormality occurrence locations within the power supply device 100 can be specified, as well as abnormality occurrence locations outside the power supply device 100.

Note that this embodiment has been described with an example of the case where the second insertion loss IL(2) and the third insertion loss IL(3) in addition to the first return loss RL(1) and the first insertion loss IL(1) are calculated based on the fourth signal SIG(4) and the source signal SIG(0), in addition to the first signal SIG(1), the second signal SIG(2), and the third signal SIG(3). However, the present invention is not limited to this.

For example, the third signal SIG(3) to the fourth signal SIG(4) may be obtained as a second return loss (RL(2)=SIG(3)/SIG(4)). The second return loss RL(2) indicates how much electric power is returned from the return electrode 240 for the output from the power supply device 100; that is, a reflection rate. Therefore, if the second return loss RL(2) is large, it can be assumed that an electric current is not properly flowing into the return electrode 240 due to, for example, a disconnection between the return electrode-connecting terminal 184 of the power supply device 100 and the return electrode 240. Also, if the return loss RL(2) is large, it can be assumed that the patient 900 and the return electrode 240 are not appropriately connected to each other.

Additionally, for example, the second signal SIG(2) to the fourth signal SIG(4) may be obtained as a fourth insertion loss (IL(4)=SIG(2)/SIG(4)). The fourth insertion loss IL(4) indicates how much electric power output from the power supply device 100 passes through the return electrode 240, the patient 900, and the treatment instrument 220 to enter the power supply device 100, that is, a passage rate. Therefore, if the fourth insertion loss IL(4) is small, it can be assumed, for example, that there is a large leakage current to an operator or other devices.

The above embodiments of the present invention encompass the following invention:

[1]

A power supply device for a treatment system to supply a high frequency electric current between an end electrode of a treatment instrument that uses high frequency power, and a return electrode that is configured to be affixed to a surface of a human body, the power supply device comprising:

a power source which generates alternating-current power;

a treatment instrument-connecting terminal for electrically connecting the treatment instrument;

an active side detection circuit which acquires a first signal for the alternating-current power output from the power source to the treatment instrument via the treatment instrument-connecting terminal, and a second signal for a power output from the power source to the treatment instrument via the treatment instrument-connecting terminal and returned from the treatment instrument to the treatment instrument-connecting terminal; and

a processor which calculates a first return loss as the second signal to the first signal and determines that an error has occurred if a variation of the first return loss, calculated per unit of time, exceeds a predetermined threshold while the calculated first return loss is increasing.

Additional advantages and modifications will readily occur to those skilled in the art. Therefore, the invention in its broader aspects is not limited to the specific details and representative embodiments shown and described herein. Accordingly, various modifications may be made without departing from the spirit or scope of the general inventive concept as defined by the appended claims and their equivalents.

1. A power supply device for a treatment system to supply a high frequency electric current between an end electrode of a treatment instrument that uses high frequency power and a return electrode that is configured to be affixed to a surface of a human body, the power supply device comprising:
a power source which generates alternating-current power;
a treatment instrument-connecting terminal for electrically connecting the treatment instrument;
a return electrode-connecting terminal for electrically connecting the return electrode;
an active side detection circuit which acquires a first signal for the alternating-current power output from the power source to the treatment instrument via the treatment instrument-connecting terminal, and a second signal for a power output from the power source to the treatment instrument via the treatment instrument-connecting terminal and returned from the treatment instrument to the treatment instrument-connecting terminal;
a passive side detection circuit which acquires a third signal for a power output from the power source to the treatment instrument via the treatment instrument-connecting terminal and passing through the return electrode to the return electrode-connecting terminal; and
a processor which calculates a return loss as the second signal to the first signal and a first insertion loss as the third signal to the first signal, and specifies an occurrence location of an abnormality based on the return loss and the first insertion loss upon occurrence of the abnormality in the treatment system,
wherein the processor determines that an error has occurred if a variation of the return loss or a variation of the first insertion loss per unit of time exceeds a predetermined threshold while the calculated return loss is increasing or while the calculated first insertion loss is decreasing.

2. The power supply device according to claim 1, wherein the processor determines
that there is an abnormality on a path between the treatment instrument-connecting terminal and the treatment instrument if the variation of the return loss is larger than a predetermined threshold, and
that there is an abnormality on a path between the return electrode and the return electrode-connecting terminal if the variation of the return loss is smaller than a predetermined threshold and the variation of the first insertion loss is smaller than a predetermined threshold.

3. (canceled)

4. (canceled)

5. The power supply device according to claim 1, wherein the processor determines that an error has occurred if the variation of the calculated return loss does not fall below a predetermined threshold or the variation of the calculated first insertion loss does not exceed a predetermined threshold after a predetermined period has passed.

6. (canceled)

7. The power supply device according to claim 1, wherein each of the active side detection circuit and the passive side detection circuit comprises only a coil, a capacitor, and a diode.

8. A treatment system comprising:
the power supply device according to claim 1;
the treatment instrument; and
the return electrode.
